(19) United States
(12) Patent Application Publication
(10) Pub. No.: US 2004/0143177 A1
(43) Pub. Date: Jul. 22, 2004

(54) Imaging table

(76) Inventors: Michael G. Falbo SR., Gladstone, MO (US); Kerry A. O'Rourke, Overland Park, KS (US)

Correspondence Address: SPENCER, FANE, BRITT & BROWNE, 1000 WALNUT STREET, SUITE 1400, KANSAS CITY, MO, 64106-2140, US

(21) Appl. No.: 10/346,218

(22) Filed: Jan. 17, 2003

(51) Int. Cl. A61B 5/05

(52) U.S. Cl. 600/407

(57) ABSTRACT

A radiolucent patient surgical support surface is provided the support surface having voids or areas of reduced support surface thickness adjacent an intended surgical field to allow reduction of the amount of X-ray energy or other imaging energy needed to produce an image of the procedure field and for observation by medical personnel.

### FIELD OF THE INVENTION

A patient imaging support surface is provided having a generally radiolucent construction. In particular the support surface of the present invention is provided with an area of reduced support surface thickness or a void in the surface which is adjacent the portion of the patient's body that is the intended area of imaging or image-guided surgery. The area of reduced support surface thickness or a void decreases the attenuation of the imaging energy or X-ray energy that is needed to produce an image.

### BACKGROUND OF THE INVENTION

In modern medicine, a technique has become widely used which is generally known as the image-guided procedure. In general, during an image-guided procedure, a patient is placed on a surgical table having a radiolucent area. During the course of the medical procedure, the positioning of medical devices and instruments within the patient is monitored by using an imaging energy source, such as X-rays. This visualization of the surgical or procedural field allows medical personnel to observe the position of the medical instruments and devices within the patient. It also allows medical personnel to determine the directions of repositioning movements of the medical instruments and/or the movements of the surgical activity being performed upon the patient. All this is accomplished without making large incisions into patients to allow the direct visual inspection of the placement of medical instruments and devices within the patient. In general, such surgical activities or procedures may be classified as percutaneous procedures and which are accomplished by performing the procedure or surgery with instruments and devices that are inserted through the skin of the patient and without the use of large incisions to provide direct access to the site of interest within the patient.

Such surgery for both diagnostic and interventional procedures is well known and includes coronary catheterization and coronary angiography, carotid stenting, percutaneous translumenal coronary angioplasty (PTCA) as well as spinal and central nervous system pain management procedures among other procedures. During these procedures, cardiologists and/or radiologists monitor the progress of the procedure through images of the operating field, typically using X-rays as the imaging energy. However, with the advancement of other imaging techniques such as magnetic resonance imaging (MRI) and computer assisted tomography (CAT) and computer tomography (CT) and with the increasing miniaturization of surgical instruments and probes, the use of imaging guided surgery is an ever expanding field.

The use of such imaging energy, in particular, X-rays in fluoroscopy, present certain potentials for harm and injury to both medical personnel and patients. High doses of radiation can result in skin burns, loss of hair and sterility. While these effects require that the dose of radiation exceed a certain threshold level, it should be appreciated that, with respect to the skin, radiation doses are additive, that is they summate, and even doses that are encountered weeks or months apart can cause damage. A dose of about 600 rad can cause abnormal redness of the skin, whereas the radiation dose of 2000 rad can cause serious skin burns. Therefore, protecting patients from harmful effects of radiation requires that the dose delivered be below the threshold dose for injury or damage. Since the highest dose delivered to a patient generally occurs on the skin at the point where the X-ray beam enters the patient, skin burns are the most frequent problem associated with current image-guided procedures.

In the early 1990s, the Food and Drug Administration (FDA) became concerned over the high radiation output of newer equipment being used in medical procedures and the length of procedure times which in some cases were as long as 325 minutes. Some skin doses during procedures were estimated to exceed 20 Gy (Gy=gray=1 Joule per kilogram). In contrast, the occupational radiation dose per year is not to exceed 0.05 Gy per year. Injuries from such radiation exposure caused the FDA to issue public health advisories in 1994 to physicians and health care administrators warning of the potential for serious skin injury during fluoroscopy procedures. The FDA outlined safety principles to make fluoroscopy safer. These principles included various suggested methods for reducing fluoroscopy exposure. The suggestion included dose monitoring techniques, moving the X-ray beam to a new skin area where fluoroscopy times exceed 30 minutes, using last-image-hold features (freeze frame) to review the image rather than using active fluoroscopy, collimating the X-ray beam to reduce the field of X-ray exposure, keeping the image intensifier close to the patient and the X-ray source as far away as possible, not removing the separator cone which forces a minimum distance between the source and the exit-beam port, using variable-pulse-rate fluoroscopy which pulses the beam at the lowest frequency suitable for the study and which can reduce dose rates dramatically. The FDA also suggested “hardening the beam” by either increasing fluoroscopic peak kilovoltage (which may reduce image contrast), or using a filter of copper, aluminum or tantalum. In summary, the FDA suggestions were, for means of specifically directing the imaging energy, or to narrow the field of the imaging energy, or to suggest techniques to reduce the total exposure to the imaging energy for both the physician and the patient. The improvement presented by the present invention—the use of areas of reduced imaging signal attenuation—was not suggested.

It will be appreciated by those skilled in the art that an additional issue is presented in image-guided procedures in which the operating field of the patient, such as the chest or abdomen, must be supported on a surface. In these procedures, the X-rays or other imaging energy must be transmitted through the patient support surface before striking the film or digital detector or other energy receiving device that allows the image of the operating field and the procedure instruments within the patient to be observed by the medical personnel. Such a patient support surface is shown in FIG. 1 and it can be appreciated that during the performance of an image-guided procedure, the imaging energy released from imaging source 13 must pass through both patient 16 and patient imaging support surface 15 prior to contacting imaging receiver 17. Depending on the type of material used to construct imaging support surface 15, the reduction in the transmission of imaging energy can be significant. For example, a 1 mm thickness of aluminum will reduce the transmission of X-rays by approximately 26 percent. If a support surface is constructed of a phenolic resin having a thickness of 12.7 mm, the reduction in X-ray transmission will be approximately 40 percent. Modern construction of patient support surfaces relies on a composite construction utilizing several different materials to combine strength with minimization of X-ray transmission loss. In the construction of such imaging support surfaces, it will be appreciated that a support surface such as that shown in FIG. 1 must be cantilevered from a support or base or other structure to allow imaging receiver 17 to be positioned opposite the imaging energy source 13 without being obstructed by structures supporting imaging support surface 15. This is accomplished by cantilevering the support surface from a base 18. However, to provide proper support for a patient and a safety factor, such cantilevered patient support surfaces should be load rated to 400 pounds or more with a required four times safety factor. Therefore, the cantilevered portion of the patient imaging support surface must be certified to support between 1200 and 1600 pounds.

Modern patient support surfaces achieve this load rating and safety factor by use of sandwich type construction which joins, for example, a foam core interior which is bonded to high-strength carbon fiber skins. Such carbon fiber/foam sandwiches provide highly radiolucent structures which are generally light weight and can provide the necessary load support required. However, for example, a patient support comprised of a sandwich having two 8 mm layers of a carbon fiber sandwich top and bottom surrounding a 15 mm foam laminate core would present a 36% reduction in X-ray transmission between the strength of the beam emanating from imaging source 13 and the strength of the beam received by receiver 17. This reduction excludes the amount of transmission loss due to the patient.

Therefore, it would be a great benefit if a patient support surface could be developed which would reduce the imaging signal attenuation or loss of transmission of energy from an imaging beam emanating from an imaging source. Such a patient support surface would provide the benefits of reducing the amount of imaging energy necessary to allow the medical personnel to view the operating field during image-guided procedures. An imaging support surface having a lower degree of imaging beam attenuation could improve procedures in two ways: (1) this approach could improve image resolution at current energy levels thereby potentially hastening the procedure and/or improving the outcome; or (2) this approach could allow the use of lower amounts of imaging energy during image-guided procedures and would permit longer periods of time for medical procedures that are image-guided. In addition, a patient support surface which reduces the attenuation of the energy in an imaging beam would provide additional safety for both patient and medical personnel by reducing the total amount of exposure of both patients and medical personnel to radiation energy. For example, burns to patients.

One aspect of the prior art should be noted, for while it presents an opening in the surface of a patient imaging support surface it would not be useful for the problem addressed by the present invention. Referring to FIG. 2, an opening 44 is shown in the area of the support surface on which rests the head of a patient. When a patient is placed face down on the support surface, this opening receives the patient's nose and face and improves patient comfort during procedures performed in the prone position. Typically, this opening can have dimensions of five inches width and six inches length. While the face opening has been offered for several years in patient support tables, the purpose is patient comfort, and thus, it incorporates several limitations which eliminate its utility as a viewing field having an area of low attenuation. Further, these limitations obscure any suggestion that such a face opening would be useful in providing an area of reduced attenuation for use with an imaging signal.

The size of the face opening is so small that an unobstructed field of view during an image-guided medical procedure could not be obtained because the opening is smaller than the receiver. If such an opening were used as a low signal attenuation support surface, the sides or edges of opening 44 would present areas or lines of poor resolution in the image that was generated. While such openings have been used in imaging support tables for many years, no use or suggestion to use face opening 44 to provide increased imaging signal transmission is known.

These preceding benefits and objects of the invention and other benefits can be obtained in an imaging patient support surface which is constructed according to the principals of the present invention which is described here and after.

### SUMMARY OF THE INVENTION

The present invention achieves the foregoing benefits and objects of the invention by providing a patient imaging support surface which comprises voids or areas of reduced thickness or areas of reduced signal attenuation in the vicinity of the particular operating field of the particular procedure being performed by medical personnel. The present invention allows the percentage of transmission of X-rays or other imaging energy being transmitted to be increased by reducing the amount of energy attenuation caused by the patient imaging support surface. This reduction in attenuation is provided by, in one embodiment of the present invention, the use of specifically located voids in the radiolucent support surface to eliminate attenuation of the imaging energy by the support surface. In another embodiment of the present invention areas of reduced support surface thickness are employed within the patient imaging support surface to reduce the amount of attenuation of the X-ray or other imaging signal. In another embodiment of the present invention tracks of reduced support surface thickness or tracks of partial voids are employed in the patient imaging support surface, the tracks following a pathway of a surgical procedure such as the path leading from a femoral blood vessel to the heart.

These areas of reduced imaging signal attenuation are achieved by a combination of features comprising the use of areas of reduced support surface thickness and/or voids in the support surface and/or the use of structural support members throughout the imaging support surface which provide greater strength to the cantilevered aspect of the imaging support surface while maintaining the support members outside the operating field of interest involved in the particular procedure.

By providing interchangeable support surfaces and by combining these features in different ways and by employing patient imaging support surfaces having localities of reduced attenuation which are positioned proximate to the portion of the patients body containing the surgical field of interest, or containing the medical devices or instruments to be viewed, the amount of energy required to provide useful images of the operating field can be reduced and the level of safety to both physician and medical personnel can be increased for most or all procedures done with image guidance.

The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention.

### DESCRIPTION OF THE DRAWINGS

Preferred embodiments of the invention, illustrative of the best modes in which the applicant has contemplated applying the principles, are set forth in the following description and are shown in the drawings and are particularly and distinctly pointed out and set forth in the appended claims.

FIG. 1 is a perspective view of a prior art imaging device and a prior art patient support surface mounted on a support frame and base;

FIG. 2 is a perspective view of a prior art imaging patient support surface unmounted from a support frame and base;

FIG. 3 is a bottom plan view of the patient imaging support of the present invention showing an area of reduced thickness positioned adjacent to an abdominal surgical field or for use during endovascular procedures and showing options for additional support structure for the embodiment in phantom lines;

FIG. 4 is a top plan view of the patient imaging support of the present invention showing a void positioned adjacent to a cardiovascular surgical field and showing options for additional support structure for the embodiment in phantom lines;

FIG. 5 is a bottom plan view of the patient imaging support of the present invention showing an area of reduced thickness positioned adjacent to the operating field for cranial, cervical, shoulder girdle and showing options for additional support structure for the embodiment in phantom lines;

FIG. 6 is a bottom plan view of the patient imaging support of the present invention showing an area of reduced thickness positioned adjacent to a cardiovascular surgical field and showing options for additional support structure for the embodiment in phantom lines;

FIG. 7 is a cross-sectional view of an embodiment of the present invention having a void to reduce attenuation of the imaging signal and having the patient positioned over the void and showing the interruption of the imaging energy created by the impinging of a chamfered edge at the perimeter of the void;

FIG. 8 is a cross-sectional view of an embodiment of the present invention having a void to reduce attenuation of the imaging signal and having the patient positioned over the void and showing the interruption of the imaging energy created by the impinging of a radius edge at the perimeter of the void;

FIG. 9 is a cross-sectional view taken along line 9-9 of FIG. 6 and showing an area of reduced imaging signal attenuation the embodiment having a top surface spanning the area of reduced attenuation and core segments having a radius edge around the area of reduced attenuation and a bottom surface on the core and showing options for additional support from embodiments in phantom lines;

FIG. 10 is a bottom plan view of another embodiment of the patient imaging support of the present invention showing an area of reduced thickness positioned adjacent to a cardiovascular surgical field and showing a track or pathway of reduced thickness leading from the portion of the imaging support adjacent the leg of the patent to the cardiovascular surgical field and showing options for additional support structure for the embodiment in phantom lines;

FIG. 11 is a top plan view of the embodiment of FIG. 10 showing the patient imaging support of the present invention and showing an area of reduced thickness and a track in phantom lines and showing options for additional support structure for the embodiment in phantom lines;

FIG. 12 is a bottom plan view of another embodiment of the patient imaging support of the present invention showing an area of reduced thickness positioned adjacent to a cranial, cervical, carotid and/or shoulder girdle surgical field and showing options for additional support structure for the embodiment in phantom lines; and

FIG. 13 is a top plan view of the embodiment of FIG. 12 showing an area of reduced thickness positioned adjacent to a cranial, cervical, carotid and/or shoulder girdle surgical field in phantom lines and showing options for additional support structure for the embodiment in phantom lines.

### DESCRIPTION OF THE PREFERRED EMBODIMENTS

As required, detailed embodiments of the present inventions are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring now to FIG. 1, a typical medical imaging apparatus and patient support to table combination 10 are shown. The combination of FIG. 1 is comprised of imaging device 12 and imaging patient support surface or imaging table 14. In general, these two devices are used together to provide a continuous or intermittent image of the positioning of surgical devices within a patient 16 during the course of an image guided diagnostic medical or surgical procedure. More particularly, a patient 16 is placed on imaging table 14 to allow the desired procedure to be performed while imaging device 12 is utilized to provide either periodic or real time observation of the surgical site and, in particular, the positioning of instruments or devices within the body of patient 16. As shown in FIG. 1, table 14 is mounted on table base 18, and the elevation of imaging table 14 is controlled by lift 20. It will be appreciated by those skilled in the art that imaging table 14 also may be equipped with motors or electromagnets, casters, pivots or rollers which allow repositioning of imaging table 14 along its longitudinal axis and its transverse axis with respect to table base 18. In this manner, imaging table 14 can be conveniently repositioned with respect to imaging source 12 without the need to change the positioning of C-arm 22 of imaging device 12. The repositionable aspects of imaging table 14 are used in combination with the repositionable aspects of imaging device 12. Imaging device 12 is positionable with respect to imaging table 14 through the use of support pivot 24 which allows rotation of imaging source 12 around a vertical or X axis.

Imaging device 12 can be additionally repositioned through the use of C-arm pivot 26 which permits repositioning of C-arm 22 around a horizontal or Y axis. It will also be appreciated by those skilled in the art that the angle of imaging source 12 is adjustable by movement of C-arm 22 in the directions indicated by arrow M and which movement is effected by gear box 28 which repositions C-arm 22 in the directions indicated by arrow M. In this manner, imaging source 13 and imaging receiver 17 are fully repositionable with respect to patient 16 and imaging table 14 to allow optimization of the position of imaging device 12 of respect to the operating field during a surgical procedure.

It is to be understood that throughout the present specification that the term imaging source is considered to encompass any form of imaging energy applied to a medical purpose including, but not limited to, for example, X-ray, light radiation from, for example, a laser, electromagnetic radiation such as, for example, radio frequency signals, nuclear magnetic resonance imaging (NCR), proton magnetic resonance imaging (PMR), positron emission tomography (PET), body radioisotope imaging generally, including gallium, iodine, and glucose isotopes, computer assisted tomography (CAT or CT), and/or magnetic resonance imaging (MRI). It is to be understood further that the term imaging receiver is considered to encompass any form of imaging energy receiving device or display device whether in fixed image or ephemeral form by which medical personnel can perceive the image of a patient produced by an imaging energy source. Such imaging receivers or displays include, but are not limited to, for example, at least film, cathode ray tube display, liquid crystal display, digital receptors, and image intensifiers,

It also is to be understood that throughout the present specification that the term radiolucent is considered to encompass the capacity of a structure or material to allow imaging energy that is applied to the structure or material to pass through the structure or material without substantial abatement or attenuation or obstruction of the imaging energy. Radiation includes without imitation, for example, electromagnetic radiation, radio frequency, light radiation, gamma radiation, ionizing radiation, electrons, alpha particles, positrons. It also should be appreciated that the present invention is useful in supporting a patient for procedures involving laser-guided procedures in which the movement of the surgical instruments within the patient's body is tracked monitoring the position of laser light emissions. It will be appreciated, however, by those skilled in the art the embodiments of the present invention that are adapted for use with electromagnetic radiation or radio frequency will require modification of some of the additional support structure shown by either moving the support structures away from the site of electromagnetic radiation or radio frequency emissions or by modifying the composition of the support structure material so that the support structure does not interfere with the electromagnetic radiation or radio frequency emissions.

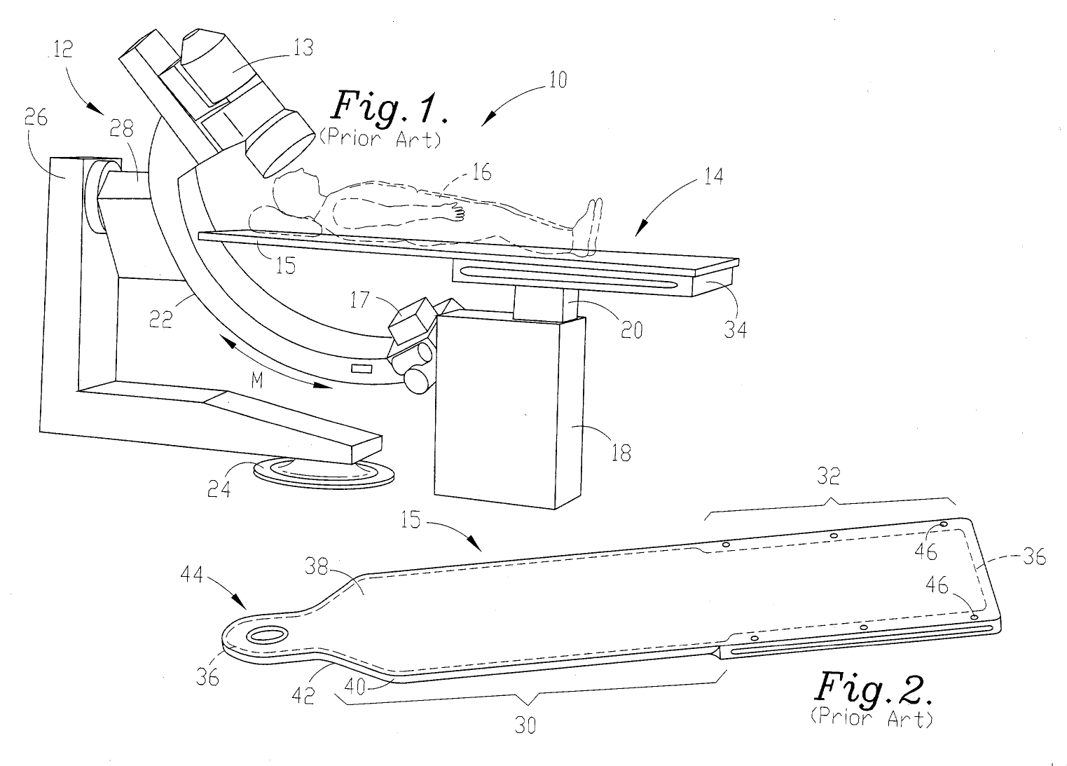

Referring now to FIG. 2, a typical prior art imaging surface 15 is shown. Imaging patient support surface 15 is a radiolucent surface which is comprised of a carbon fiber sandwich or epoxy or decorative foam laminate or polypropylene or phenolic resin or carbon fiber/foam combination Lexan® or polycarbonate or acrylic polymer or a combination of these materials. In general, such imaging support surfaces 15 are comprised of two general sections; a radiolucent area 30 and a structural support portion 32 which is connected to imaging table support frame 34 (FIG. 1) and which may or may not be radiolucent. In some prior art devices a frame 36 can be provided which extends from structural support portion 32 to provide structural support to radiolucent area 30. A support frame 36 allows radiolucent area 30 to be cantilevered from table support frame 34 thereby eliminating any obstruction in radiolucent area 30 of support surface 15 which might inhibit the movement and operation of imaging device 12. Alternatively, if the materials used in the construction of support surface 15 are sufficiently strong, a frame 36 can be eliminated. However, such frameless construction can result in a reduction in the percentage of imaging energy transmitted through the stronger materials.

While the structure and composition of structural support portion 32 and frame 36 can vary widely, it is important that radiolucent area 30 be comprised of a material or sandwich of materials which permits as much of the energy emanating from imaging source 13 (FIG. 1) as is possible to pass through radiolucent area 30. It is necessary that the energy be well sensed by imaging receiver 17 (FIG. 1) to provide medical personnel with as detailed an image as possible of the surgical area of interest. In the construction of such patient support imaging surfaces 15, it is necessary that the radiolucent material selected to form radiolucent area 30 and the materials selected to form frame 36 be sufficiently strong so a load of 400 or more pounds with a three to four times safety factor can be supported by radiolucent area 30 as it is cantilevered from table support frame 34 (FIG. 1) and structural support portion 32. This requirement that such a substantial weight be supported by radiolucent area 30 has, in the prior art devices, necessitated that a trade off be made between structural strength and radiolucence of radiolucent area 30. As previously described in the background of the invention, this results in the use of higher energy levels emanating from imaging source 13 than might otherwise be needed to view an area of interest were the patient able to be presented in unsupported fashion to imaging source 13 such as is the case with a chest X-ray.

Generally, the composition of the prior art patient imaging support surface 15 is that of a three-layer “sandwich” which comprises a top surface which is typically comprised of carbon fiber or carbon fiber and epoxy or phenolic resin. Top surface 38 is adhered to a core 40 which in prior art imaging support surfaces is comprised of a structural foam core which contributes to the strength and rigidity of the imaging surface. The bottom face of core 40 is attached to bottom surface 42 which typically, in prior art imaging surfaces, is a second carbon fiber or carbon and epoxy or phenolic resin skin. It is also typical of prior art imaging support surfaces that a head opening 44 be provided. Head opening 44 is a void in imaging support surface 15 which is only so large as to allow the face of the patient to be placed in depression or void 44 when the patient is lying face down on support surface 15 during procedures. As described in the Background of the Invention, the face opening that is common in many support surfaces is so small an area that it could not be used as an area of reduced attenuation particularly since the edges of the opening would be so close to the field of diagnosis or surgery that they would obscure the image produced. Support surface 15 is attached to table support frame 34 (FIG. 1) through the use of attachment voids 46 through which a bolt or other connector is passed to secure imaging support surface 15 to table support frame 34.

As previously described in the background of the invention, it would be advantageous if less support material was presented by imaging support surface 15 as this would reduce the amount of attenuation of the energy emanating from image source 30 during medical procedures. Less material also would allow for the amount of energy required from imaging source 13 (FIG. 1) to be reduced thereby presenting a safer situation for both patient and medical personnel during medical procedures. The present invention accomplishes these goals, generally, by providing voids or areas of reduced thickness in imaging support surface 15 which, when located in the area of the surgical procedure to be performed, enable the reduction of the amount of energy that is released from imaging source 30 in order to provide a clear view of the medical procedure field to the medical personnel during the course of a medical procedure.

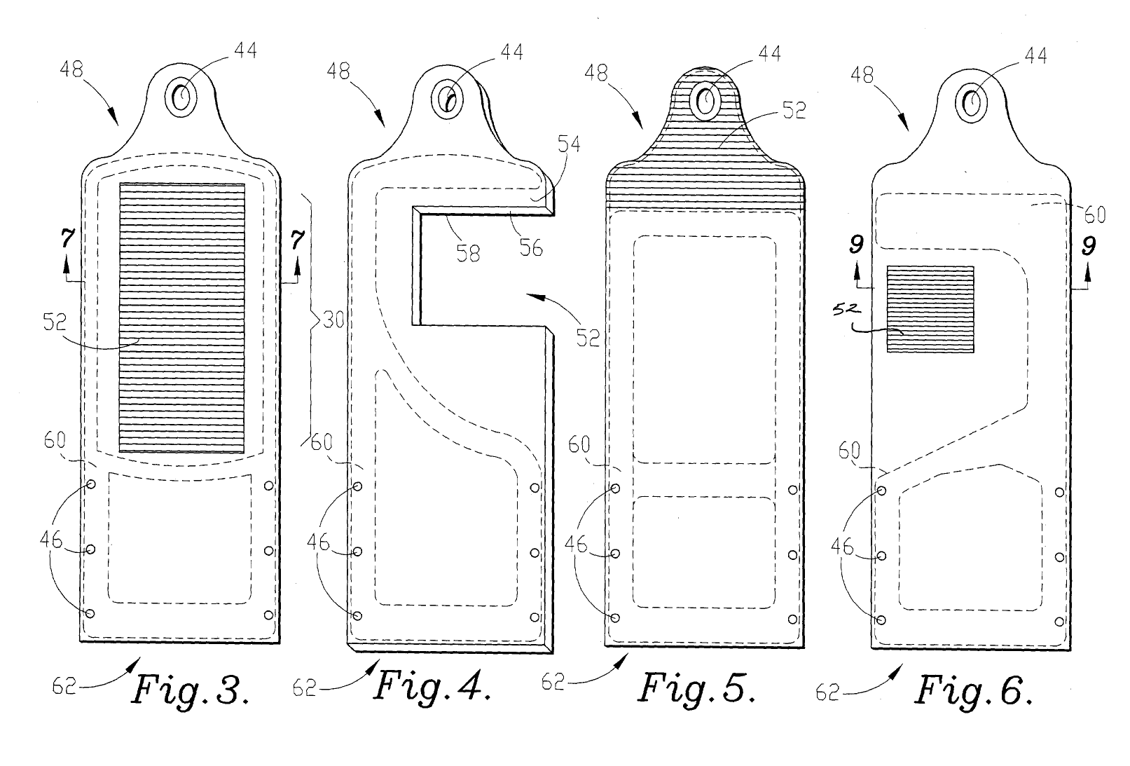

Referring now FIGS. 3, 4, 5 and 6, various embodiments of the present invention are shown which enable the objects and benefits of the present invention to be realized. Referring now to FIG. 3, an imaging support 48 is shown which embodies the present invention. (In FIGS. 3-9, the imaging support containing the present invention is numbered as reference number 48 to distinguish it from the prior art imaging support surface of FIG. 2 which was numbered as 15.) In FIG. 3, area of reduced attenuation 52 is provided which, according to the present invention, is an area which can be a void in imaging support 48 or, alternatively, area of reduced attenuation 52 can be an area of reduced thickness in imaging support 48 or, alternatively, area 52 can be a portion of imaging support 48 which is comprised of only top surface 54 (FIG. 9) with the portion or core 56 (FIG. 9) and bottom surface 58 (FIG. 9) corresponding to area of reduced attenuation 52 being absent. Still referring to FIG. 3, reduced attenuation area 52 in the embodiment of FIG. 3 is an area which corresponds to the medical procedure field for endovascular/abdominal diagnostic and surgical procedure.

In operation, the patient's face or back of patient's head is placed into head opening 44 and the shoulders, torso, abdomen and legs are supported on imaging support 48. As previously described, area of reduced attenuation 52 provides for greater transmission of the energy from imaging source 13 (FIG. 1) than does a conventional patient imaging support surface such as that shown in FIG. 2. The conventional imaging support surface 15 of FIG. 2 would typically be comprised of a top surface 38 of approximately 8 mm of thin carbon fiber sandwich, a core 40 of approximately 15 mm of foam core and a bottom surface 42 of approximately 8 mm of thin carbon fiber sandwich. This prior art composition of imaging support surface 15 results in a substantial attenuation of the energy which is generated by imaging source 13. Typically, 8 mm of thin carbon fiber sandwich will provide a transmission for X-rays of 98 percent (or 2 percent attenuation the X-ray). A 15 mm thickness of foam laminate will provide 80 percent transmission of an X-ray (or 20 percent attenuation of X-rays). Therefore, a typical prior art construction of imaging support surface 15 which is comprised of 8 mm of thin carbon fiber sandwich as a top surface 38 and bottom surface 42 and 15 mm of foam core 40 will result in approximately a 24 percent attenuation of X-rays or only a 76 percent transmission of the X-rays which are generated by an X-ray source.

By contrast, in the present invention, in the situation in which area of reduced attenuation 52 is a void, zero percent attenuation of the imaging source signal occurs, and the only attenuation of the signal results from the patient's body and the operating instruments and paraphernalia which are within the medical procedure field. In the embodiment of the present invention in which area of reduced attenuation 52 is comprised of only a top surface 54 comprised of, for example, 8 mm of thin carbon fiber sandwich, the attenuation resulting from area 52 is only a 2 percent transmission loss of the imaging signal as 8 mm of thin carbon fiber sandwich provides 98 percent transmission of the imaging signal (based on the transmission of X-rays through carbon fiber sandwich). Therefore, with the present invention, a surgical team is able to achieve substantially higher rates of signal transmission from an imaging source 13 (FIG. 1). The medical procedure can be conducted with a substantial reduction in the exposure of the patient and the medical team to the energy produced by the imaging source. In the case previously described based on top and bottom surfaces of 8mm of thin carbon fiber surrounding a 15 mm foam film core, the reduction in radiation exposure in the case of X-rays is approximately 22 and 24 percent. Thus, the present invention provides the surgeon or radiologist with the benefit of less exposure to imaging source radiation (of whatever type) or the ability to safely extend the length of time needed for a procedure, or the ability to reduce the amount of protective shielding worn by medical personnel or the option of achieving clearer images having better resolution by maintaining the energy strength of the imaging source used during the procedure. Depending on the priorities of the medical procedure and/or the medical team involved in the procedure, the present invention provides a variety of benefits and advantages which can be selectively used by the medical personnel to obtain the particular benefit which is most useful to the particular type of medical procedure being conducted. For example, where it is critical to have a clearer image of the medical procedure field with better resolution, the physician may elect to maintain the convention imaging strength and to use the benefits of the present invention to obtain an improved image of the field. Alternatively, in a difficult procedure, the physician may determine the greater benefit is achieved from reducing the imaging source strength thereby allowing more time to conduct the procedure while exposing the patient and medical personnel to the same amount or lesser amount of X-ray radiation or other imaging energy than would be received using a prior art imaging support surface 15.

An additional benefit is achieved through use of the present invention as the construction of imaging support surface 48 having areas of reduced attenuation 52 which are intended to correspond to the medical procedure field of a particular procedure by allowing for increased internal framing support to be used in the construction of imaging support 48. Still referring to FIG. 3, the framing 60 which can be included in imaging support 48 is shown in phantom lines. Frame 60 in FIG. 3 is shown surrounding area of reduced attenuation 52. Frame 60 can be designed in multiple ways and placed within inches of area 52 in the present invention due to the specific placement of area 52 proximate to the locus of surgery. In the prior art support, such close framing 60 would not be possible as it would interfere with other medical procedures have a different procedural locus or field of surgery. In addition, frame 60 is substantially larger than the framing which could be included in prior art imaging support surface 15 (FIG. 2) and can be comprised of materials which are more radiopaque than previous materials used in prior art imaging support surfaces 15 but which provide greater strength. Thus, by the use of additional frame 60 in imaging support 48 greater strength can be provided to radiolucent portion 30 of imaging support 48 which extends out of table support frame 34 (FIG. 1). This additional framing allows the use of materials for the construction of top surface 54, core 56, and bottom surface 58 (FIG. 9) that cost less than, for example, carbon fiber and which provide less strength but which can be used in the present invention in constructing imaging support 48 due to the increased framing. Since each area of reduced attenuation 52 is intended to be adjacent to a particular operating or surgical field, the designated areas 52 define those portions of imaging support 48 from which radiopaque materials and/or interfering support structure should be excluded. The remainder of imaging support 48 can include radiopaque materials and/or interfering support structure such as are shown in phantom lines by support structure 60 as those locations are not apart of the surgical or operating field. In a prior art imaging support surface 15 the inclusion of additional radiopaque materials and/or interfering support structure 60 would not be permitted as such prior art imaging support surfaces 15 are intended to be applicable to all, or at least a wide variety of, medical procedures and the additional support structure 60 would interfere with many surgical or operating fields in such prior art general purpose support surfaces 15.

Referring now to FIG. 4, an alternative embodiment of the present invention is shown. In FIG. 4 imaging support 48 is provided with an alternative shaping and location of area of reduced attenuation 52. In the embodiment of FIG. 4, area of reduced attenuation 52 is shown as a void created by the removal of top surface 54, core 56 and bottom surface 58 in the area of reduced attenuation 52. This location of area 52 is intended for use in cardiovascular procedures, therefore, the complete absence of support structure in the area corresponding to the operating field of cardiovascular surgery provides a surgeon or radiologist with a completely unobstructed view of the area of interest. Again, as shown in FIG. 4, additional and more substantial support structures can be included in the present invention 48 as shown in phantom lines by the frame structure 60 which can be included in imaging support 48 and which can intrude into areas of imaging support 48 which would not be permitted in prior art constructions. As shown in FIG. 4, frame 60 can extend from foot 62 toward head opening 44 and can surround the area of reduced attenuation 52, thereby providing increased support to imaging support 48 and providing the option of using less costly materials in the construction of imaging support 48.

Referring now to FIG. 5, another embodiment of the present invention is provided wherein area of reduced attenuation 52 comprises the end of imaging support 48 which is opposite foot 62. In the embodiment of FIG. 5, the area of reduced attenuation 52 is incorporated into imaging support 48 in the area which would correspond to the operating field for cranial, cervical, carotid shoulder girdle procedures. Again, by to examination of FIG. 5, it will be appreciated that as area of reduced attenuation 52 is provided in an area of imaging support 48 which corresponds to the operating field for specific surgical procedures, that additional framing 60, which extends over a greater area than could be permitted in prior art, support surface 15, is included to better support the patient on imaging support 48. Again, the additional framing 60 allows for the use of alternate materials which can be less expensive and which may provide a lesser contribution to the required load which must be supported by imaging support 48.

Referring now to FIG. 6, an alternative embodiment of imaging support 48 is shown in which area of reduced attenuation 52 is an area of reduced thickness which is achieved by the elimination of core 56 and bottom surface 58 in the area of reduced attenuation 52. In the embodiment of FIG. 6, area of reduced attenuation 52 is, as is with the embodiment of FIG. 4, intended for cardiovascular surgeries. However, instead of area of reduced attenuation 52 being a void as in the embodiment of FIG. 4, in the embodiment of FIG. 6 area of reduced attenuation 52 is an area of reduced thickness of imaging support 48. In FIG. 6, only top surface 54 is present in area of reduced attenuation 52. This construction provides the benefits previously described in the discussion of the embodiment shown in FIG. 3 and, in the case of cardiovascular surgery allows for improved visualization of the heart and major arteries and veins and can improve the surgical outcomes in procedures involving plaque, identification and/or plaque removal or stent placement. Again, as previously described in the discussion of alternate embodiments of imaging support 48, the embodiment shown in FIG. 6 permits the inclusion of substantial amounts of framing 60 which surround the area of reduced attenuation 52 of imaging support 48 and permits the use of alternate materials in the construction of imaging support 48.

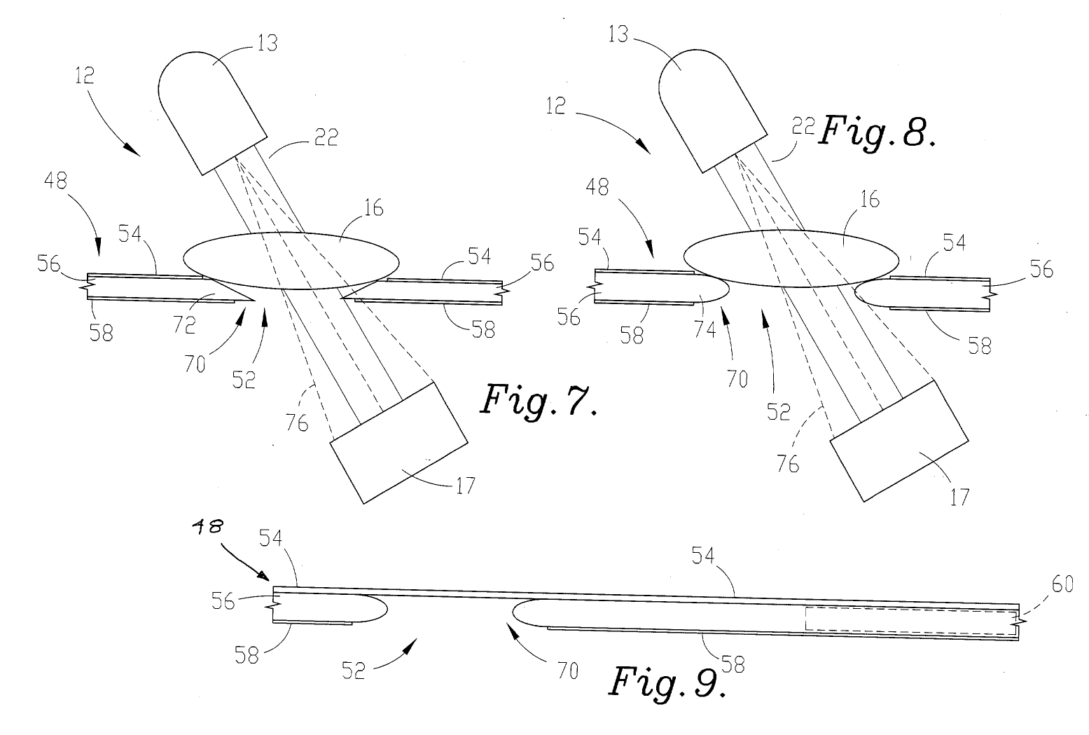

Referring now to FIGS. 7 and 8, additional details of the construction of reduced attenuation area 52 will be described. When providing an area of reduced attenuation 52, which can be either a void or an area of reduced thickness, edges 70 that surround the area of reduced attenuation 52 and which, to some degree, will impinge upon the outer perimeter of the medical procedure field as it is visualized by an imaging device 12 (FIG. 1) can be shaped so as to reduce the degree of obstruction or deterioration of the image which is experienced at the outer perimeter of the area of reduced attenuation 52. In FIGS. 7 and 8, two alternative shapes of edges 70 are presented. In FIG. 7, a chamfered edge 72 is shown and in FIG. 8 a radius edge 74 is shown. An examination and comparison of FIG. 7 with FIG. 8 shows that a radius edge 74, in some situations, may provide less obstruction of the perimeter of the medical procedure field occurs using radius edge 74 as compared to chamfered edge 72. It will be appreciated by those skilled in the art that chamfered edge 72 could be placed at the bottom of support 48 as is shown in FIG. 7, or chamfered edge 72 could be placed at the top of support 48. In either case, the amount of obstruction caused by chamfered 72 would be similar. Also a square or right angle edge can be used with the invention. In FIG. 7, an imaging source 13 is positioned above patient 16 and is emitting energy such as an X-ray which is traveling through patient 16 and being received by imaging receiver 17. As is shown in FIG. 7, as X-ray energy 76 travels through patient 16, it is obstructed by chamfered edge 72 which enters into the portion of the surgical field being visualized when imaging device 12 is canted on an angle as is shown in FIG. 7 and 8. By contrast, radius edge 74 which is shown in FIG. 8 substantially limits the amount of obstruction which is occurring at the perimeter of the surgical field and therefore represents a preferred embodiment of the edges 70 which are used in the present invention.

Referring now to FIG. 9, a cross-sectional view of the embodiment of the present invention shown in FIG. 6 will be discussed. In FIG. 9, a form of construction of imaging support 48 is shown having top surface 54 which spans across area of reduced attenuation 52 and is supported on either side of area 52 by core 56. Depending on the type of materials employed, a bottom surface 58 can be included in the construction of imaging support 48. As is shown in FIG. 9, bottom surface 58 would terminate near area 52 and would not span area 52 as does top surface 54 in an embodiment of the present invention in which an area of thinness is provided to create area of reduced attenuation 52. Alternatively, FIGS. 7 and 8 show an embodiment of imaging support 48 in cross-section view in which a void is provided to form area of reduced attenuation 52 and both top surface 54 and bottom surface 58 terminate and do not span area 52.

Referring now to FIGS. 10 and 11, an alternative embodiment of the present invention is shown in which an area of reduced attenuation is provided in image support 48. In the embodiment of FIGS. 10 and 11, the area of reduced attenuation corresponds to the operating field for cardiovascular surgery and extends downwardly to include the leg area of the patient within the area of reduced attenuation 52. This track or pathway of reduced attenuation which extends downwardly to include the leg of the patient is provided to encompass viewing of the entire pathway of the patient's vascular system which is involved in catheterization of the patient though the blood vessels of the leg. As shown in FIG. 11, the top surface of this embodiment of the present invention can be a flat surface which presents to the physician the area of reduced attenuation 52 in diagrammatic fashion such as outlining or dotted lines. The diagrammatic display of area of reduced attenuation 52 can be placed either on the support surface itself or on 1s a pad which is placed on top of the support surface. It will be appreciated by those skilled in the art that alternative embodiments of such tracks or pathways of reduced attenuation as shown in FIGS. 10 and 11 could be provided for other surgical procedures such as a catheterization in which the point of insertion is the arm.

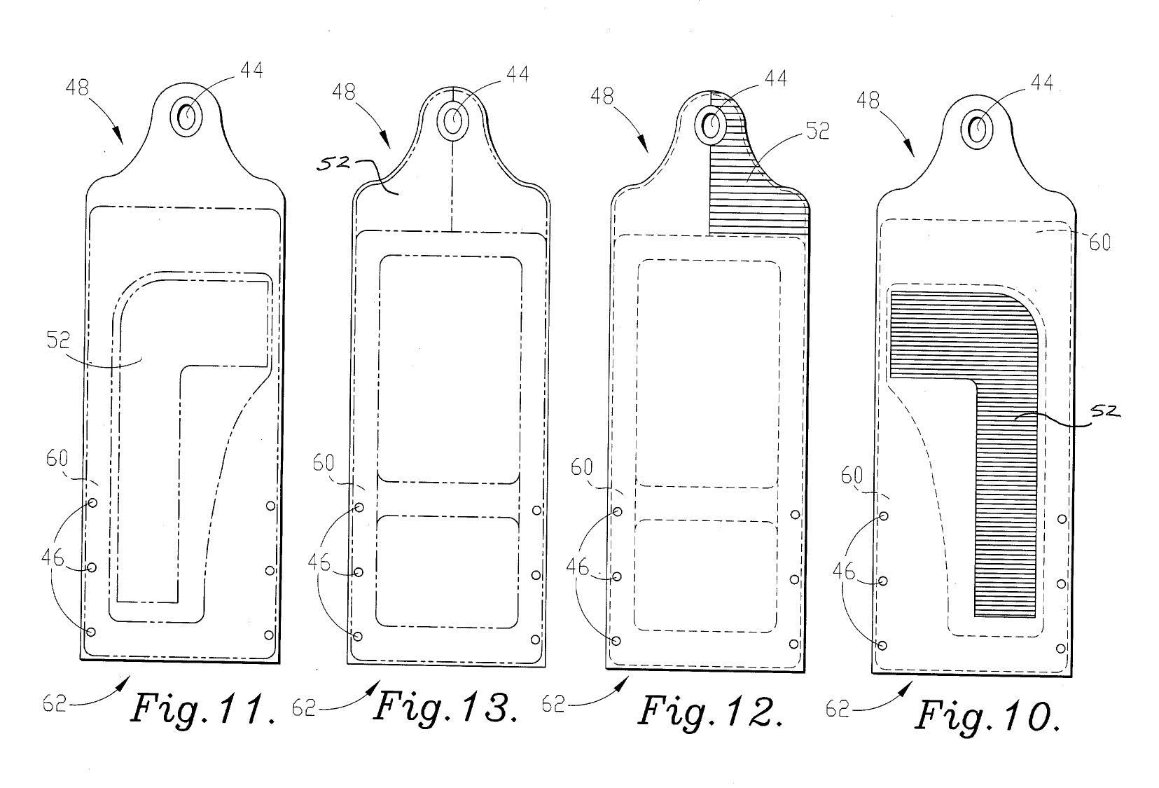

Referring now to FIGS. 12 and 13, an embodiment of the present invention is shown in which the area of reduced attenuation 52 is provided in the neck and shoulder area. This area of reduced attenuation is adjacent to the operating field for cranial, cervical, carotid and shoulder girdle surgeries. As is shown in phantom lines, additional support structure 60 can be included thereby allowing the use of different, or the use of reduced strength materials and the support surface to further increase the radiolucence of the support surface 48 in addition to the increased transmission provided by area of reduced transmission 52.

In the foregoing description, certain terms have been used for brevity, clearness and understanding; but no unnecessary limitations are to be implied therefrom beyond the requirements of the prior art, because such terms are used for descriptive purposes and are intended to be broadly construed. Moreover, the description and illustration of the inventions is by way of example, and the scope of the inventions is not limited to the to exact details shown or described.

Certain changes may be made in embodying the above invention, and in the construction thereof, without departing from the spirit and scope of the invention. It is intended that all matter contained in the above description and shown in the accompanying drawings shall be interpreted as illustrative and not meant in a limiting sense.

Having now described the features, discoveries and principles of the invention, the manner in which the inventive imaging support surface is constructed and used, the characteristics of the construction, and advantageous, new and useful results obtained; the new and useful structures, devices, elements, arrangements, parts and combinations, are set forth in the appended claims.

It is also to be understood that the following claims are intended to cover all of the generic and specific features of the invention herein described, and all statements of the scope of the invention which, as a matter of language, might be said to fall therebetween.

Having thus described the invention what is claimed as new and desired to be secured by Letters Patent is as follows:

1. A generally radio-opaque support surface for holding the body of a patient thereon during imaging of the patient the surface comprising:
at least one area of radiolucence positioned on said support to be adjacent to an area of the body which is the locus of a medical procedure.

2. The support as claimed in claim 1 wherein said area of radiolucence comprises a void in said support.

3. The surface as claimed in claim 2 wherein said void is defined by an edge comprising a curved surface.

4. The surface as claimed in claim 2 wherein said void is defined by an edge comprising a chamfered surface.

5. The surface as claimed in claim 1 wherein said area of radiolucence comprises an area of reduced thickness of said support.

6. A support for holding the body of a patient thereon during imaging of the patient the surface comprising:
a generally radiolucent support comprising a top surface and a bottom surface; and
at least one area of increased radiolucence on said support said area being positioned to be adjacent to an area of the body upon which a medical procedure is performed.

7. The surface as claimed in claim 6 wherein said area of increased radiolucence comprises a void in said support.

8. The surface as claimed in claim 6 wherein said area of increased radiolucence comprises a void in said top surface or said bottom surface.

9. The surface as claimed in claim 8 wherein said void is defined by an edge comprising a curved surface.

10. The surface as claimed in claim 8 wherein said void is defined by an edge comprising a chamfered surface.

11. The surface as claimed in claim 6 wherein said area of increased radiolucence comprises an area of reduced thickness of said support.

12. The surface as claimed in claim 6 wherein said medical procedure is a cardiovascular procedure.

13. The surface as claimed in claim 6 wherein said medical procedure is an angioplasty procedure.

14. The surface as claimed in claim 6 wherein said medical procedure is an angiography procedure.

15. The surface as claimed in claim 6 wherein said medical procedure is a femoral catheterization procedure.

16. The surface as claimed in claim 6 wherein said medical procedure is a cranial procedure.

17. The surface as claimed in claim 6 wherein said medical procedure is a cervical spine procedure.

18. The surface as claimed in claim 6 wherein said medical procedure is a thoracic spine procedure.

19. The surface as claimed in claim 6 wherein said medical procedure is a procedure on the shoulder girdle.

20. The surface as claimed in claim 6 wherein said medical procedure is a procedure on the abdomen.

21. A radiolucent support for holding the body of a patient thereon while conducting imaging of the patient comprising:
a generally radiolucent top surface,
a generally radiolucent bottom surface,
at least one area of increased radiolucence on said radiolucent support, and
a load supporting fame proximate to said at least one area of increased radiolucence said frame joining said surfaces.

22. The surface as claimed in claim 21 wherein said at least one area of increased radiolucence is positioned to be adjacent the medical procedure field of a patient's body during the performance of an image-guided medical procedure.

23. The surface as claimed in claim 22 wherein said area of increased radiolucence comprises a void in said support.

24. The surface as claimed in claim 23 wherein said void is defined by an edge comprising a curved surface.

25. The surface as claimed in claim 23 wherein said void is defined by an edge comprising a chamfered surface.

26. The surface as claimed in claim 22 wherein said area of increased radiolucence comprises a void in said top surface or said bottom surface.

27. The surface as claimed in claim 22 wherein said area of increased radiolucence comprises an area of reduced thickness of said support.

28. A method of increasing the transmission of imaging energy through a patient imaging support table during an image-guided medical procedure comprising:
providing an imaging patient support surface, reducing the thickness of a portion of said support surface said portion being positioned on said support surface to be adjacent an area of the patient which is the locus of an image-guided medical procedure, and
directing an imaging energy source through said reduced thickness portion to provide an image for observation during an image-guided medical procedure.

29. The surface as claimed in claim 28 wherein said medical procedure is a cardiovascular procedure.

30. The surface as claimed in claim 28 wherein said medical procedure is an angioplasty procedure.

31. The surface as claimed in claim 28 wherein said medical procedure is an angiography procedure.

32. The surface as claimed in claim 28 wherein said medical procedure is a femoral catheterization procedure.

33. The surface as claimed in claim 28 wherein said medical procedure is a cranial procedure.

34. The surface as claimed in claim 28 wherein said medical procedure is a cervical spine procedure.

35. The surface as claimed in claim 28 wherein said medical procedure is a thoracic spine procedure.

36. The surface as claimed in claim 28 wherein said medical procedure is a procedure on the shoulder girdle.

37. The surface as claimed in claim 28 wherein said medical procedure is a procedure on the abdomen.

38. A method of increasing the transmission of imaging energy through a patient imaging support table during an image-guided medical procedure comprising:
providing an imaging patient support surface, and
positioning a void in said support surface said void being positioned on said support surface to be adjacent an area of the patient which is the locus of an image-guided medical procedure, and
directing an imaging energy source through said void to provide an image for observation during an image-guided medical procedure.

39. The surface as claimed in claim 38 wherein said medical procedure is a cardiovascular procedure.

40. The surface as claimed in claim 38 wherein said medical procedure is an angioplasty procedure.

41. The surface as claimed in claim 38 wherein said medical procedure is an angiography procedure.

42. The surface as claimed in claim 38 wherein said medical procedure is a femoral catheterization procedure.

43. The surface as claimed in claim 38 wherein said medical procedure is a cranial procedure.

44. The surface as claimed in claim 38 wherein said medical procedure is a cervical spine procedure.

45. The surface as claimed in claim 38 wherein said medical procedure is a thoracic spine procedure.

46. The surface as claimed in claim 38 wherein said medical procedure is a procedure on the shoulder girdle.

47. The surface as claimed in claim 38 wherein said medical procedure is a procedure on the abdomen.

48. A method of performing an image-guided medical procedure on a patient comprising:
providing a patient imaging support surface having in said surface an area of reduced imaging energy attenuation,
supporting the patient on said surface,
orienting the patient on said surface to position said area of reduced attenuation adjacent an intended medical procedure field,
adjusting an imaging source to transmit imaging energy through the patient and through said area of reduced attenuation, and
receiving said transmitted imaging energy on an imaging receiver.
